(19) United States
(12) Patent Application Publication
(10) Pub. No.: US 2022/0315980 A1
(43) Pub. Date: Oct. 6, 2022

(54) COMPOSITION FOR URINE GLUCOSE DETECTION AND SYSTEM FOR IDENTIFYING URINE GLUCOSE BY USING SAME

(71) Applicant: Young Wook KIM, Seoul (KR)

(72) Inventor: Young Wook KIM, Seoul (KR)

(21) Appl. No.: 17/640,179

(22) PCT Filed: Aug. 31, 2020
(86) PCT No.: PCT/KR2020/011611

§ 371 Date: Mar. 3, 2022

(30) Foreign Application Priority Data
Sep. 10, 2019 (KR) 10-2019-0111941

(51) Int. Cl. C12Q 1/54; B01L 3/00; C12Q 1/26

(52) U.S. Cl. CPC C12Q 1/54; B01L 3/5023; C12Q 1/26; B01L 2300/069; B01L 2300/023; E03D 9/00

(57) ABSTRACT

A composition for urine glucose detection includes a fixing agent, an indicator and glucose oxidase, wherein the fixing agent includes one or more selected from the group consisting of a soap base, gelatin, and natural fatty acids.

### TECHNICAL FIELD

The present invention relates to a composition for detecting urine glucose and a system for identifying urine glucose using the same, and more particularly, to a user-friendly composition for detecting urine glucose, which is capable of identifying urine glucose through a non-invasive process, and a system for identifying urine glucose using the same.

### BACKGROUND ART

Diabetes mellitus (known as diabetes) is a type of metabolic disease in which an amount of insulin secretion is insufficient or insulin secretion is not normally functioning, and its incidence is increasing due to aging and changes in eating habits. Patients suffering from diabetes need to check blood glucose several times a day in order to manage and control a blood glucose level.

Conventionally, a measurement method in which blood collected by pricking a fingertip with a needle is put into a blood glucose monitoring device was used. However, such an invasive measurement method caused great pain and discomfort in the blood collection process that was performed several times a day, and had an economical burden of continuously purchasing consumables for blood collection.

Accordingly, there is a demand for the development of non-invasive technology that can easily detect glucose in the living body.

### DISCLOSURE

### Technical Problem

To solve the above-described problems, the present invention is directed to providing a user-friendly composition for detecting urine glucose, which can be used to determine whether urine glucose is abnormal without a separate blood collection process, and a system for identifying urine glucose using the same.

### Technical Solution

A composition for detecting urine glucose according to an embodiment of the present invention includes a fixing agent, an indicator and glucose oxidase, wherein the fixing agent consists of one or more selected from the group consisting of a soap base, a gelatin and a natural fatty acid.

In addition, the indicator is one selected from the group consisting of Thymol blue, Methyl orange, Methyl red, Bromothymol blue, Cresol red, and phenolphthalein.

In addition, the glucose oxidase and the fixing agent are included in a weight ratio of 1:10 to 2000.

In addition, the fixing agent is a mixture of two materials selected from the group consisting of a soap base, a gelatin and a natural fatty acid. When a mixture of the soap base and the gelatin is selected as the fixing agents, the weight ratio of the soap base and the gelatin is set to be 1:0.01 to 100, when a mixture of the soap base and the natural fatty acid is selected as the fixing agent, the weight ratio of the soap base and the natural fatty acid is set to be 1:01 to 10, and when a mixture of the natural fatty acid and the gelatin is selected as the fixing agent, the weight ratio of the natural fatty acid and the gelatin is set to 1:0.01 to 100.

In addition, the composition for detecting urine glucose is used by being processed into a solid or gel type.

The system for identifying urine glucose according to one embodiment of the present invention includes a toilet into which cleaning water for detecting urine glucose generated by a composition for detecting urine glucose is provided, a color sensor for sensing the color of the cleaning water for detecting urine glucose corresponding to a user's urine provided to the toilet, and a communication module for providing color information of the cleaning water for detecting urine glucose sensed by the color sensor to a networked smart home appliance, wherein the composition for detecting urine glucose includes a fixing agent, an indicator and glucose oxidase, and the fixing agent consists of one or more selected from the group consisting of a soap base, a gelatin and a natural fatty acid.

In addition, the composition for detecting urine glucose is processed into a solid state, and provided in a water tank for providing cleaning water to the toilet.

In addition, the composition for detecting urine glucose is processed into a gel type, and supplied by a separate supply unit.

In addition, the system for identifying urine glucose further includes a user recognition unit for recognizing the user, and the communication module provides user information recognized by the user recognition unit to the smart home appliance.

In addition, the toilet is a toilet seat or a urinal.

### Advantageous Effects

As described above, the present invention can provide a user-friendly composition for detecting urine glucose and a system for identifying urine glucose using the same, which can determine an abnormality in urine glucose without a separate blood collection process.

In addition, the present invention can provide a composition for detecting urine glucose and a system for identifying urine glucose using the same, which can remove the discomfort felt during the blood collection process and alleviate the economic burden of purchasing consumables for blood collection.

In addition, the present invention can provide a composition for detecting urine glucose and a system for identifying urine glucose using the same, which is able to be installed in a toilet and thus can be easily used by anyone in everyday life.

In addition, the present invention can provide a system for identifying urine glucose that includes a user recognition unit to manage individual users.

In addition, the present invention can provide a system for identifying urine glucose, which has a function in conjunction with a smart home appliance to provide health management information to users based on the results of checking the user's urine glucose.

### DESCRIPTION OF DRAWINGS

FIG. 1 is a diagram illustrating a system for identifying urine glucose according to one embodiment of the present invention.

FIG. 2 is a diagram of a toilet seat to which the system for identifying urine glucose according to one embodiment of the present invention is applied.

FIG. 3 is a diagram of a urinal to which the system for identifying urine glucose according to one embodiment of the present invention is applied.

### MODES OF THE INVENTION

Hereinafter, embodiments related to the present invention are illustrated in the drawings and will be described in detail through detailed description. However, it should be understood that the present invention is not limited to embodiments disclosed herein, and includes all modifications, equivalents or alternatives within the spirit and scope of the present invention.

In addition, the term “comprise,” “include,” “consist of,” or “have” used herein means that, unless specifically stated otherwise, a corresponding component may be included, and it should be understood that other components may be further included, rather than excluded.

Hereinafter, a composition for detecting urine glucose and a system for identifying urine glucose using the same according to embodiments of the present invention will be described with reference to drawings related to the embodiments of the present invention.

The composition for detecting urine glucose according to an embodiment of the present invention is used to detect a glucose level in a user's urine through a color change, and may include a fixing agent, an indicator and glucose oxidase.

The fixing agent serves to form the physical structure of the composition and physically support the composition, and also serves to determine the concentration of an enzyme reacting with glucose later by adjusting the amount of the dissolved glucose oxidase, which is the enzyme.

Here, the fixing agent may consist of one or more materials selected from the group consisting of a soap base, a gelatin and a natural fatty acid.

The soap base is a mixture of basic materials used to produce a soap, and for example, may be prepared by adding an alkaline solution such as an aqueous sodium hydroxide solution or an aqueous potassium hydroxide solution to a fatty acid.

The gelatin is a type of induced protein obtained by thermally treating a natural protein, collagen, and the like, which constitute the skin, tendons and cartilage of an animal.

The natural fatty acid is a natural fatty acid, and may be, for example, myristic acid, palmitic acid, stearic acid, lauric acid, arachidic acid, lignoceric acid, palmitoleic acid, oleic acid, linoleic acid or arachidonic acid, but the present invention is not limited thereto.

The indicator has various colors depending on an acidity (or pH) changed by the reaction with urine glucose, and thus may serve to intuitively inform a user of the current urine glucose level through a color change.

The indicator used in one embodiment of the present invention may consist of one or more selected from the group consisting of Thymol blue, Methyl orange, Methyl red, Bromothymol blue, Cresol red, and phenolphthalein.

The glucose oxidase may serve to convert glucose into gluconic acid and hydrogen peroxide (H₂O₂).

Accordingly, the composition may be used to detect and quantify glucose in a user's urine, and the higher the urine glucose level, the higher the acidity of the corresponding solution. The color of the solution is changed due to the indicator according to the change in acidity. Therefore, it is possible to easily check and detect the user's urine glucose through the color change.

In one example, the composition for detecting urine glucose may show a first color when the user's urine glucose is in a normal range, and show a second color when the user's urine glucose is in an abnormal range.

A weight ratio of the glucose oxidase and the fixing agent may be variously changed, but these materials are preferably mixed in a weight ratio of 1:10 to 2000. That is, when the amount of the fixing agent is larger than the above-described proportion, the solubility of the glucose oxidase is lowered, thereby lowering the sensitivity to the detection of urine glucose. On the contrary, when the amount of the fixing agent is smaller than the above-described proportion, the glucose oxidase is excessively and quickly diffused to the solution, which likely shortens the life of the product.

In addition, the composition for detecting urine glucose composed as described above may be processed into a solid or gel type as needed and then used.

Meanwhile, the composition for detecting urine glucose may be classified into the following embodiments according to the type of fixing agent and the type of indicator.

### First Embodiment

A composition for detecting urine glucose according to a first embodiment may include a soap base; any one indicator selected from the group consisting of Thymol blue, Methyl orange, Methyl red, and phenolphthalein; and glucose oxidase.

In the first embodiment, to satisfy the required solubility and product life of the glucose oxidase, the weight ratio of the glucose oxidase and the fixing agent was set to 1:100 to 1000.

In addition, since the composition according to the first embodiment has strong basicity due to the soap base, the Bromothymol blue and Cresol red indicators were not able to realize a desired color change under the corresponding conditions, so they were excluded from the corresponding composition.

### Second Embodiment

A composition for detecting urine glucose according to a second embodiment may include a gelatin; any one indicator selected from the group consisting of Thymol blue, Methyl orange, Methyl red, Bromothymol blue, Cresol red, and phenolphthalein; and glucose oxidase.

In the second embodiment, to satisfy the required solubility and product life of the glucose oxidase, the weight ratio of the glucose oxidase and the fixing agent was set to 1:10 to 2000.

In addition, since the composition according to the second embodiment is neutral, there was no limitation to the type of indicator, and it was confirmed that the fixing efficiency was higher than that of the soap base.

### Third Embodiment

A composition for detecting urine glucose according to a third embodiment may include a natural fatty acid; any one indicator selected from the group consisting of Thymol blue, Methyl orange, Methyl red, and phenolphthalein; and glucose oxidase.

In the third embodiment, to satisfy the required solubility and product life of the glucose oxidase, the weight ratio of the glucose oxidase and the fixing agent was set to 1:10 to 2000.

In addition, since the composition according to the third embodiment has strong basicity due to the natural fatty acid, the Bromothymol blue and Cresol red indicators were not able to realize a desired color change under the corresponding conditions, so they were excluded from the corresponding composition, and it was confirmed that the fixing efficiency was higher than that of the soap base.

### Fourth Embodiment

A composition for detecting urine glucose according to a fourth embodiment employs a soap base and a gelatin as fixing agents; any one indicator selected from the group consisting of Thymol blue, Methyl orange, Methyl red, Bromothymol blue, Cresol red, and phenolphthalein; and glucose oxidase.

In the fourth embodiment, to satisfy the required solubility and product life of the glucose oxidase, the weight ratio of the glucose oxidase and the fixing agents was set to 1:100 to 1000.

In addition, the proportion of the gelatin may determine the overall pH, affecting indicator selection, and in consideration of the fact that when there is more gelatin, the solubility of the enzyme is lowered and the product life is extended, the weight ratio of the soap base and the gelatin was set to 1:0.01 to 100.

### Fifth Embodiment

A composition for detecting urine glucose according to a fifth embodiment may employ a soap base and a natural fatty acid as fixing agents, one indicator selected from the group consisting of Thymol blue, Methyl orange, Methyl red, Bromothymol blue, Cresol red, and phenolphthalein; and glucose oxidase.

In the fifth embodiment, to satisfy the required solubility and product life of the glucose oxidase, the weight ratio of the glucose oxidase and the fixing agents was set to 1:100 to 1000.

In addition, in consideration of the fact that when the amount of the natural fatty acid increases, the solubility of the enzyme is affected and the reliability/sensitivity of the product is affected, the weight ratio of the soap base and the natural fatty acid was set to 1:0.1 to 10.

### Sixth Embodiment

A composition for detecting urine glucose according to a sixth embodiment may employ a gelatin and a natural fatty acid as fixing agents; one indicator selected from the group consisting of Thymol blue, Methyl orange, Methyl red, Bromothymol blue, Cresol red, and phenolphthalein; and glucose oxidase.

In the sixth embodiment, to satisfy the required solubility and product life of the glucose oxidase, the weight ratio of the glucose oxidase and the fixing agents was set to 1:10 to 2000.

The proportion of the gelatin may determine overall pH, affecting indicator selection, and in consideration of the fact that when there is more gelatin, the solubility of the enzyme is lowered and the product life is extended, the weight ratio of the natural fatty acid and the gelatin was set to 1:0.01 to 100.

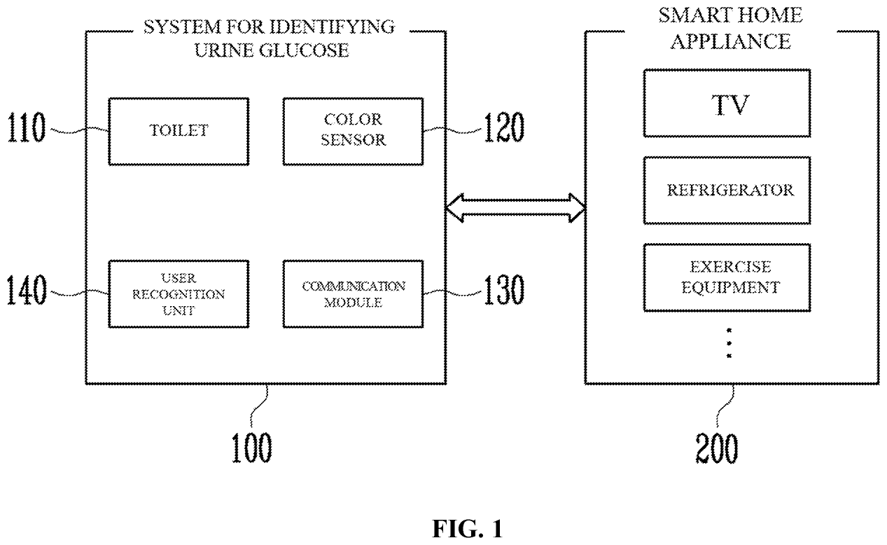

FIG. 1 is a diagram illustrating a system for identifying urine glucose according to one embodiment of the present invention.

A system for identifying urine glucose 100 according to one embodiment of the present invention is used to easily detect a user's urine glucose using the composition for detecting urine glucose described above, and may be operated in conjunction with various types of smart home appliances 200 utilizing the Internet of Things (IoT).

Referring to FIG. 1, the system for identifying urine glucose 100 according to one embodiment of the present invention may include a toilet 110, a color sensor 120 and a communication module 130, and may further include a user recognition unit 140 according to the form of the embodiment.

The toilet 110 may receive cleaning water for detecting urine glucose generated through the composition for detecting urine glucose. Afterward, when a user who needs to check urine glucose urinates in the toilet 110, cleaning water for detecting urine glucose located in the toilet 110 reacts with glucose in the urine, resulting in a color corresponding to the amount of glucose.

The color sensor 120 may sense a color of the cleaning water for detecting urine glucose corresponding to the user's urine provided to the toilet 110.

That is, by sensing the color change of the cleaning water for detecting urine glucose according to the user's urine glucose level, it is possible to determine whether the current user's urine glucose level is in a normal or abnormal range.

For example, when the color of the cleaning water for detecting urine glucose reacting with the urine is a first color, it may be determined that the user's urine glucose level is in the normal range, and when the color of the cleaning water for detecting urine glucose reacting with the urine is a second color, it may be determined that the user's urine glucose level is in the abnormal range.

In one example, the color sensor 120 may recognize the color of the cleaning water by a camera module or a photodiode. However, the present invention is not limited thereto, and the color sensor 120 may employ various types of color sensors and image sensors, which are known in the art.

In addition, the color sensor 120 may be installed by integrating with the toilet 110 to sense the color of the cleaning water, or separately installed on the ceiling of a bathroom in which the toilet 110 is located to sense the color of the cleaning water.

The communication module 130 may provide the color information of the cleaning water for detecting urine glucose sensed by the color sensor 120 to a networked smart home appliance 200. Such a communication module 130 may be designed integrally with the toilet 110, and integrated with the toilet 110.

The smart home appliance 200 may receive the color information of the cleaning water for detecting urine glucose from the communication module 130 of the system for identifying urine glucose 100, and determine the user's health condition based on the information. That is, according to the color information, it is possible to determine whether the urine glucose level is in a normal or abnormal range based on the color information, and health management information may be provided to the user according to the above-described determination result.

In one example, when the urine glucose level of the user is confirmed to be in the abnormal range, an exercise for managing blood glucose or a diet for managing blood glucose may be suggested to the user. In addition, more accurate measurement of a urine glucose level may be suggested to the user or a hospital visit may be recommended.

The smart home appliance 200 is a home appliance enabling information exchange with a different device or system via a network such as the Internet, and in one example, may include a TV, a refrigerator or exercise equipment, and the like, furnished in the home. However, the type of smart home appliance 200 is not limited thereto, and can be expanded to various types of other home appliances.

The user recognition unit 140 is for recognizing a user of the system for identifying urine glucose 100, and may be implemented by various user recognition devices.

For example, the user recognition unit 140 may be configured to include a fingerprint sensor, an iris recognition sensor, a facial recognition sensor, or a voice recognition sensor. However, the method for user recognition is not limited thereto, and various biometric technologies may be employed.

The user recognition unit 140 may be integrally designed with the toilet 110, and integrated with the toilet 110.

The user information recognized by the above-described user recognition unit 140 may be provided to the smart home appliance 200 via the communication module 130. That is, it is possible to specify a user who currently uses the system for identifying urine glucose 100 through the user recognition unit 140.

In addition, the smart home appliance 200 may individually manage the numerical data on urine glucose for each user in response to such user information, and provide user-customized health management information by utilizing user information.

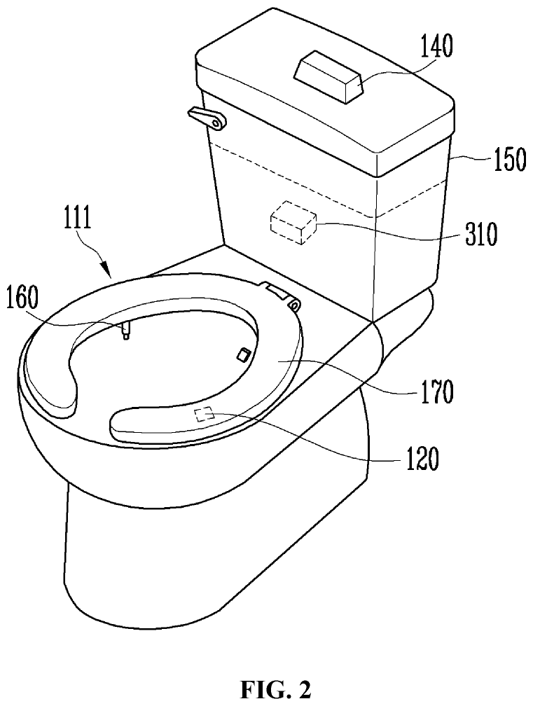

FIG. 2 is a diagram of a toilet seat to which the system for identifying urine glucose according to one embodiment of the present invention is applied.

A toilet seat 111 according to one embodiment of the present invention may hold cleaning water for detecting urine glucose in an internal reservoir for cleaning water.

This cleaning water for detecting urine glucose may be supplied by a water tank 150. To this end, a composition for detecting urine glucose 310 may be processed into the form of a solid block and provided into the water tank 150. Accordingly, cleaning water for detecting urine glucose may be generated in the water tank 150, and the cleaning water for detecting urine glucose may be provided to the reservoir for cleaning water in the toilet seat 111.

Meanwhile, in another embodiment, the composition for detecting urine glucose may be processed into a gel type and directly supplied into the toilet seat 111 by a separate supply unit 160. For example, the supply unit 160 may be installed in the toilet seat 111 to discharge the gel-type composition for detecting urine glucose in a certain amount. Accordingly, general cleaning water in the toilet seat 111 supplied from the water tank 150 may be converted into cleaning water for detecting urine glucose by the composition for detecting urine glucose.

Such a supply unit 160 may be operated in conjunction with the above-described user recognition unit 140. For example, after a specific user is recognized by the user recognition unit 140, the supply unit 160 may automatically supply a certain amount of the composition for detecting urine glucose, which is required for one cycle of detection, to the reservoir for cleaning water in the toilet seat 111.

The supply unit 160 may be implemented in the form of a dispenser capable of dispensing the gel-type composition for detecting urine glucose, but the present invention is not limited thereto.

In addition, FIG. 2 shows an embodiment in which the supply unit 160 is installed in the toilet seat 111, but the location of the supply unit 160 is not limited thereto. In one example, the supply unit 160 may be installed in the water tank 150.

Meanwhile, a color sensor 120 may be installed in the toilet seat 111 while not being in contact with cleaning water in the toilet seat 111, and in one example, as shown in FIG. 2, the color sensor 120 may be installed on the lower side of the toilet cover 170.

In addition, as shown in FIG. 2, in one example, the user recognition unit 140 may be installed on the water tank 150, but the present invention is not limited thereto, and the location of the user recognition unit 140 may be variously designed.

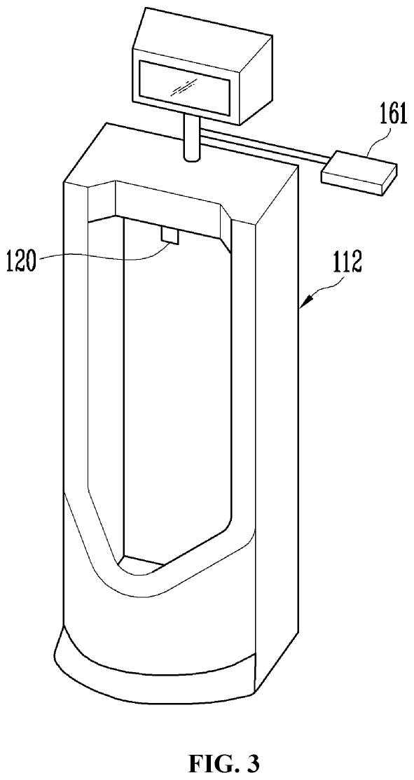

FIG. 3 is a diagram of a urinal to which the system for identifying urine glucose according to one embodiment of the present invention is applied.

Referring to FIG. 3, cleaning water for detecting urine glucose generated through the composition for detecting urine glucose may be provided in a urinal 112 according to one embodiment of the present invention.

In one example, a supply unit 161 accommodating the composition for detecting urine glucose processed into a gel type may be separately installed to supply a certain amount of the composition for detecting urine glucose, required for urine glucose detection, to the urinal 112.

Accordingly, since the cleaning water supplied to the urinal 112 is mixed with the composition for detecting urine glucose, a user can check urine glucose using the urinal 112.

Meanwhile, a color sensor 120 may be installed in the urinal 112 while not being in contact with cleaning water therein, and in one example, as shown in FIG. 3, may be installed on the upper side in the urinal 112.

In addition, while not separately shown in FIG. 3, a separate user recognition unit 140 for user recognition may be further installed.

It should be understood by those of ordinary skill in the art that the embodiments disclosed herein can be easily modified into other specific forms without changing the technical spirit or essential features of the present invention. Therefore, it should be interpreted that the embodiments described above are exemplary in all aspects, and are not limitative. The scope of the present invention is defined by the appended claims, rather than the detailed description, and encompasses all modifications and alterations derived from meanings, the scope and equivalents of the appended claims.

1. A composition for detecting urine glucose, comprising:
a fixing agent;
an indicator; and
glucose oxidase,
wherein the fixing agent consists of one or more selected from the group consisting of a soap base, a gelatin, and a natural fatty acid.

2. The composition of claim 1, wherein the indicator is one or more selected from the group consisting of Thymol blue, Methyl orange, Methyl red, Bromothymol blue, Cresol red, and phenolphthalein.

3. The composition of claim 1, wherein the glucose oxidase and the fixing agent are comprised in a weight ratio of 1:10 to 2000.

4. The composition of claim 1, wherein the fixing agent is a mixture of two materials selected from the group consisting of a soap base, a gelatin, and a natural fatty acid,
when a mixture of the soap base and the gelatin is selected as the fixing agent, the weight ratio of the soap base and the gelatin is set to be 1:0.01 to 100,
when a mixture of the soap base and the natural fatty acid is selected as the fixing agent, the weight ratio of the soap base and the natural fatty acid is set to be 1:01 to 10, and
when a mixture of the natural fatty acid and the gelatin is selected as the fixing agent, the weight ratio of the natural fatty acid and the gelatin is set to 1:0.01 to 100.

5. The composition of claim 1, wherein the composition for detecting urine glucose is used by being processed into a solid or gel type.

6. A system for identifying urine glucose using a composition for detecting urine glucose,
a toilet into which cleaning water for detecting urine glucose generated by a composition for detecting urine glucose is provided;
a color sensor for sensing the color of the cleaning water for detecting urine glucose corresponding to a user's urine provided to the toilet; and
a communication module for providing color information of the cleaning water for detecting urine glucose sensed by the color sensor to a networked smart home appliance,
wherein the composition for detecting urine glucose comprises
a fixing agent;
an indicator; and
glucose oxidase, and
the fixing agent consists of one or more selected from the group consisting of a soap base, a gelatin and a natural fatty acid.

7. The system of claim 6, wherein the composition for detecting urine glucose is processed into a solid state, and provided in a water tank for providing cleaning water to the toilet.

8. The system of claim 6, wherein
the composition for detecting urine glucose is processed into a gel type, and supplied by a separate supply unit.

9. The system of claim 6, wherein the system for identifying urine glucose further comprises
a user recognition unit for recognizing the user, and
the communication module provides
user information recognized by the user recognition unit to the smart home appliance.

10. The system of claim 6, wherein the toilet is a toilet seat or a urinal.
